(19) United States
(12) Patent Application Publication
(10) Pub. No.: US 2024/0390527 A1
(43) Pub. Date: Nov. 28, 2024

(54) CROSSLINKED HYDROGELS WITH TUNABLE RADIOPACITY FOR MEDICAL APPLICATIONS

(71) Applicant: Boston Scientific Scimed, Inc., Maple Grove, MN (US)

(72) Inventors: Yen-Hao Hsu, Shrewsbury, MA (US); Sasha Viola Rios, Minneapolis, MN (US); Nicolas Ball-Jones, Minneapolis, MN (US); Joseph Thomas Delaney, JR., Minneapolis, MN (US); Cristian Parisi, Boston, MA (US)

(73) Assignee: Boston Scientific Scimed, Inc., Maple Grove, MN (US)

(21) Appl. No.: 18/670,474

(22) Filed: May 21, 2024

Related U.S. Application Data
(60) Provisional application No. 63/504,385, filed on May 25, 2023.

(51) Int. Cl. A61K 49/04

(52) U.S. Cl. CPC A61K 49/0442; A61K 49/0457; A61K 2121/00

(57) ABSTRACT

In some aspects the present disclosure provides a radiopaque, reactive copolymer comprising at least one hydrophilic polymer segment having a plurality of hydrophilic polymer segment ends, a plurality of iodinated polymer segments covalently linked to the plurality of hydrophilic polymer segment ends, and a plurality of reactive moieties covalently linked to the iodinated polymer segments. In other aspects, the present disclosure pertains to a system for forming a hydrogel composition that comprises (a) such a radiopaque, reactive copolymer and (b) a nucleophilic compound, as well as to a crosslinked reaction product of the same. In further aspects, the present disclosure pertains to a method of treatment comprising administering to a subject a mixture that comprises (a) such a radiopaque, reactive copolymer and (b) a nucleophilic compound, under conditions such that the nucleophilic compound and the radiopaque, reactive copolymer crosslink after administration.

### CROSS-REFERENCE TO RELATED APPLICATIONS

This application claims the benefit of U.S. Provisional Patent Application Ser. No. 63/504,385 filed on May 25, 2023, the disclosure of which is incorporated herein by reference.

### FIELD

The present disclosure relates radiopaque hydrogels and to crosslinkable systems for forming radiopaque hydrogels, among other aspects. The radiopaque hydrogels and crosslinkable systems for forming the same are useful, for example, in various medical applications.

### BACKGROUND

Bioresorbable hydrogels with rapid crosslinking reaction rate in vivo, known by the trade name of SpaceOAR®, have become a prominent biomaterial and obtained clinical success in creating the space between prostate and rectum, tremendously improving patient safety during the cancer therapies. A further improvement based on this application is that some of 8-Arm PEG branches are functionalized with 2,3,5-triiiodobenzamide (TIB) groups, replacing part of the activated ester end groups, succinimidyl glutarate (SG), in order to provide intrinsic radiopacity to the hydrogels themselves for CT-visibility. This hydrogel, known by the trade name of SpaceOAR Vue®, is the next generation of SpaceOAR® for prostate medical applications.

While the above approach is effectual, some issues arise as a result of the incorporation of the TIB functional group. First, in order to functionalize TIB on 8-arm PEG, one succinimidyl glutarate (SG) binding site is sacrificed for each functionalized arm. Moreover, the entire functionalization process involves multiple steps, typically five steps, from commercially available hydroxyl-terminated 8-arm PEG to its functionalized form with two different end groups (TIB and SG groups). This complex process of synthesizing the 8-arm PEG results in a significant increase of the product cost, decreased hydrogel persistence, and difficulties in product quality control. Furthermore, each added TIB group occupies one arm of the star-PEG, reducing capacity and efficiency of the crosslinking reaction. Lastly, SG groups can start their degradation in acidic pH environments, which could potentially cause longer gel times and faster dissipation of crosslinked hydrogels in vivo.

For these and other reasons, alternative strategies for forming iodine-labelled crosslinked hydrogels that provide enhanced radiopacity while maintaining crosslink density per polymer molecule are desired.

### SUMMARY

The present disclosure provides an alternative approach to that described above.

In some aspects the present disclosure provides a radiopaque, reactive copolymer comprising at least one hydrophilic polymer segment having a plurality of hydrophilic polymer segment ends, a plurality of iodinated polymer segments covalently linked to the plurality of hydrophilic polymer segment ends, and a plurality of reactive moieties covalently linked to the iodinated polymer segments.

In some embodiments, the at least one hydrophilic polymer segment comprises a single hydrophilic polymer segment, and the plurality of hydrophilic polymer segment ends comprise first and second ends of the single hydrophilic polymer segment. In some of these embodiments, the first end of the single hydrophilic polymer segment is covalently linked to a first end of a first iodinated polymer segment of the plurality of iodinated polymer segments, the second end of the single hydrophilic polymer segment is covalently linked to a first end of a second iodinated polymer segment of the plurality of iodinated polymer segments, a first reactive moiety of the plurality of reactive moieties is covalently linked to a second end of the first iodinated polymer segment, and a second reactive moiety of the plurality of reactive moieties is covalently linked to a second end of the second iodinated polymer segment.

In some embodiments, the at least one hydrophilic polymer segment comprises a plurality of hydrophilic polymer segments, and the radiopaque, reactive copolymer comprises a plurality of polymer arms linked to a core region, wherein the polymer arms each comprise one of the plurality hydrophilic polymer segments, one of the plurality of iodinated polymer segments, and one of the plurality of reactive moieties. In some of these embodiments, a first end of the one of the plurality of hydrophilic polymer segments is covalently linked to the core region, a second end of the one of the plurality of hydrophilic polymer segments is covalently linked to a first end of the one of the plurality of iodinated polymer segments, and the one of the plurality of reactive moieties is covalently linked to a second end of the one of the plurality of iodinated polymer segments. In some of these embodiments, the core region comprises a polyol residue or a silsesquioxane.

In some embodiments, which can be used in conjunction with the above aspects and embodiments, the reactive moiety comprises an electrophilic group.

In some embodiments, which can be used in conjunction with the above aspects and embodiments, the reactive moiety comprises a succinimide ester group, an imidazole ester group, an imidazole carboxylate group, or a benzotriazole ester group.

In some embodiments, which can be used in conjunction with the above aspects and embodiments, the at least one hydrophilic polymer segment comprises one or more monomers selected from ethylene oxide, propylene oxide, N-vinyl pyrrolidone and an oxazoline monomer.

In some embodiments, which can be used in conjunction with the above aspects and embodiments, the at least one hydrophilic polymer segment contains between 40 and 4000 monomer residues.

In some embodiments, which can be used in conjunction with the above aspects and embodiments, the plurality of iodinated polymer segments each contain between 2 and 50 monomer residues.

In some embodiments, which can be used in conjunction with the above aspects and embodiments, the plurality of iodinated polymer segments each comprise a residue of one or more iodinated cyclic ester monomers. In some of these embodiments, the iodinated cyclic ester monomer is selected from an iodinated lactone and an iodinated 1,4-dioxane-2,5-dione compound.

In some embodiments, which can be used in conjunction with the above aspects and embodiments, the plurality of iodinated polymer segments is iodinated polyester segments in which at least one iodinated moiety is covalently attached to a polyester backbone of each of the iodinated polyester segments. In some of these embodiments, the iodinated moiety comprises a monocyclic or multicyclic aromatic structure that is substituted with one or more iodine groups, and may optionally be further substituted with one or more hydroxyl groups and/or one or more C₁-C₄-hydroxyalkyl groups.

In some embodiments, which can be used in conjunction with the above aspects and embodiments, the plurality of iodinated polymer segments each comprise residues of an iodinated cyclic ester monomer, wherein an iodinated moiety is covalently attached to at least one ring carbon of a cyclic ester portion of the iodinated cyclic ester monomer. In some of these embodiments, the iodinated moiety comprises a monocyclic or multicyclic aromatic structure that is substituted with one or more iodine groups, and may optionally be further substituted with one or more hydroxyl groups and/or one or more C₁-C₄-hydroxyalkyl groups.

In other aspects, the present disclosure pertains to a system for forming a hydrogel composition that comprises (a) a nucleophilic compound and (b) a radiopaque, reactive copolymer in accordance with any of the above aspects and embodiments. In some of these embodiments, nucleophilic compound is a polyamino compound.

In some embodiments, the system comprises a first composition that comprises the nucleophilic compound and a second composition that comprises the radiopaque, reactive polymer. In some of these embodiments, the system further comprises an accelerant composition.

In some embodiments, which can be used in conjunction with the above aspects and embodiments, the system comprises one or more additional agents selected from therapeutic agents, imaging agents, colorants, tonicity adjusting agents, suspension agents, wetting agents, and pH adjusting agents.

In some embodiments, which can be used in conjunction with the above aspects and embodiments, the system further comprises a delivery device.

In additional aspects, the present disclosure pertains to radiopaque crosslinked hydrogel composition comprising a crosslinked reaction product of (a) a nucleophilic compound and (b) a radiopaque, reactive copolymer in accordance with any of the above aspects and embodiments. In some of these embodiments, the radiopaque crosslinked hydrogel composition further comprises one or more additional agents selected from therapeutic agents, imaging agents, colorants, tonicity adjusting agents, suspension agents, wetting agents, and pH adjusting agents.

In further aspects, the present disclosure pertains to a method of treatment comprising administering to a subject a mixture that comprises (a) a nucleophilic compound and (b) a radiopaque, reactive copolymer in accordance with any of the above aspects and embodiments, under conditions such that the nucleophilic compound and the radiopaque, reactive copolymer crosslink after administration. In some of these embodiments, the mixture comprises one or more additional agents selected from therapeutic agents, imaging agents, colorants, tonicity adjusting agents, suspension agents, wetting agents, and pH adjusting agents.

Potential benefits associated with the present disclosure include one or more of the following: radiocontrast is maintained, crosslink density is enhanced, and in vivo persistence is obtained.

The above and other aspects, embodiments, features and benefits of the present disclosure will be readily apparent from the following detailed description.

### BRIEF DESCRIPTION OF THE DRAWINGS

FIG. 1 schematically illustrates a method of forming a linear radiopaque, reactive copolymer, in accordance with an embodiment of the present disclosure.

FIG. 2 schematically illustrates a method of forming a multi-arm radiopaque, reactive copolymer, in accordance with an embodiment of the present disclosure.

FIG. 3 schematically illustrates a method of forming a multi-arm radiopaque, reactive copolymer, in accordance with another embodiment of the present disclosure.

FIG. 4 schematically illustrates a method of forming a radiopaque crosslinked hydrogel, in accordance with an embodiment of the present disclosure.

FIG. 5 schematically illustrates a method of forming a radiopaque crosslinked hydrogel, in accordance with an embodiment of the present disclosure.

FIG. 6 shows various iodinated 1,4-dioxane-2,5-dione compounds for use in the present disclosure.

FIG. 7 illustrates a delivery device, in accordance with an embodiment of the present disclosure.

### DETAILED DESCRIPTION

The present disclosure pertains to radiopaque, reactive copolymers including radiopaque, reactive linear copolymers and radiopaque, reactive multi-arm copolymers. As discussed below, the radiopaque, reactive copolymers have tunable radiopacity without loss of reactive groups.

Radiopaque, reactive copolymers in accordance with the present disclosure include copolymers that comprise one or more hydrophilic polymer segments having at least two hydrophilic polymer segment ends, an iodinated polymer segment linked to each of the hydrophilic polymer segment ends, and a reactive moiety that is linked to each iodinated cyclic anhydride residue.

Radiopaque, reactive linear copolymers in accordance with the present disclosure include polymers that comprise a linear hydrophilic polymer segment, an iodinated polymer segment linked to each end of the linear hydrophilic polymer segment and a reactive moiety that is linked to each iodinated polymer segment.

In some embodiments, the radiopaque, reactive linear copolymers comprise a linear hydrophilic polymer segment having first and second ends, an iodinated polymer segment having a first end covalently linked to each of the first and second ends of the hydrophilic polymer segment, and a reactive moiety that is covalently linked to a second end of each iodinated polymer segment.

Radiopaque, reactive multi-arm copolymers in accordance with the present disclosure also include polymers that comprise a plurality of copolymer arms linked to a core region. The copolymer arms each comprise a hydrophilic polymer segment linked to the core region, an iodinated polymer segment linked to each hydrophilic polymer segment, and a reactive moiety that is covalently linked to each iodinated polymer segment. Radiopaque, reactive multi-arm copolymers include copolymers having two, three, four, five, six, seven, eight, nine, ten, fifteen, twenty, or more arms.

In some embodiments, the radiopaque, reactive multi-arm copolymers comprise a plurality of copolymer arms linked to a core region, the copolymer arms each comprising a hydrophilic polymer segment that has first and second ends, the first end of the hydrophilic polymer segment covalently linked to the core region, an iodinated polymer segment having first and second ends, the first end of the iodinated polymer segment covalently linked to the second end of the hydrophilic polymer segment, and a reactive moiety that is covalently linked to the second end of the iodinated polymer segment.

It is noted that the radiopacity of the above copolymers can be tuned by changing the length of the iodinated polymer segment which may contain from 2 to 50 iodinated monomer residues, for example ranging anywhere from 2 to 3 to 5 to 10 to 15 to 20 to 30 to 40 to 50 iodinated monomer residues (in other words, ranging between any two of the preceding numerical values). Moreover, in some embodiments, the iodinated polymer segment may be replaced by a single iodinated monomer residue.

In some embodiments, the reactive moiety comprises an electrophile and may be selected, for example, from succinimide ester groups, imidazole ester groups, imidazole carboxylate groups and benzotriazole ester groups, among other possibilities.

In some embodiments, the reactive moiety may be covalently linked to the iodinated polymer segments of the radiopaque, reactive multi-arm copolymers through a hydrolysable ester group. For instance, the reactive moiety may comprise a diester. In particular examples, the diester may be selected from a malonic-acid-based diester, a succinic-acid-based diester, a glutaric-acid-based diester, an adipic-acid-based diester, and a diglycolic acid-based diester. In certain embodiments, a succinimide group is linked to one ester of the diester and the other ester of the diester is linked to an iodinated polymer segment. More particular examples of such reactive moieties include a succinimidyl malonate moiety, a succinimidyl succinate moiety, a succinimidyl glutarate moiety, a succinimidyl adipate moiety, and a succinimidyl diglycolate moiety.

Although iodinated polymer segments are described above, in other embodiments, the iodinated polymer segments can be replaced by a single iodinated monomer residue.

Hydrophilic polymer segments can be selected from any of a variety of synthetic, natural, or hybrid synthetic-natural hydrophilic polymer segments. Examples of hydrophilic polymer segments include those that are formed from one or more hydrophilic monomers selected from ethylene oxide, propylene oxide, N-vinyl pyrrolidone, oxazoline monomers (e.g., oxazoline and 2-alkyl-2-oxazolines, for instance, 2-(C₁-C₆ alkyl)-2-oxazolines (including various isomers), such as 2-methyl-2-oxazoline, 2-ethyl-2-oxazoline, 2-propyl-2-oxazoline, 2-isopropyl-2-oxazoline, 2-n-butyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following copolymer segments: polyether segments including poly(alkylene oxide) segments such as poly(ethylene oxide) (PEO) (also referred to as polyethylene glycol or PEG) segments, poly(propylene oxide) segments, poly(ethylene oxide-co-propylene oxide) segments, poly(N-vinyl pyrrolidone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, protein segments or polysaccharide segments. Polymer segments for use in the multi-arm polymers of the present disclosure typically contain between 10 and 1000 monomer units.

As previously noted, in the case of radiopaque, reactive multi-arm copolymers, the copolymer arms extend from a core region. In certain of these embodiments, the core region comprises a residue of a polyol comprising two or more hydroxyl groups, which is used to form the copolymer arms. In certain beneficial embodiments, the core region comprises a residue of a polyol that contains two, three, four, five, six, seven, eight, nine, ten, eleven, twelve or more hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl) cresols, among others. Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 10 to 1000 units in length.

In other embodiments, the core region comprises a silsesquioxane. A silsesquioxane is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise copolymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

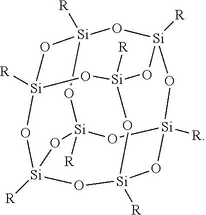

In the present disclosure, the R groups comprise the copolymer arms described herein.

Radiopaque, reactive linear copolymers in accordance with the present disclosure can be formed from hydroxy-terminated precursor linear polymers. Likewise, radiopaque, reactive multi-arm copolymers in accordance with the present disclosure can be formed from hydroxy-terminated precursor multi-arm polymers having arms that comprise one or more hydroxyl end groups.

For example, a hydroxy-terminated precursor linear hydrophilic polymer or a hydroxy-terminated precursor multi-arm hydrophilic polymer may be used as a multi-functional initiator for ring-opening polymerization with an iodinated cyclic monomer (also referred to as an iodinated cyclic monomer) though which an iodinated polymer segment can be formed. As previously noted, the radiopacity of the resulting copolymer can be tuned by changing the length of the iodinated polymer segment which may contain from 2 to 40 iodinated cyclic monomer residues. Moreover, in some embodiments, the iodinated polymer segment may be replaced by a single iodinated cyclic monomer residue.

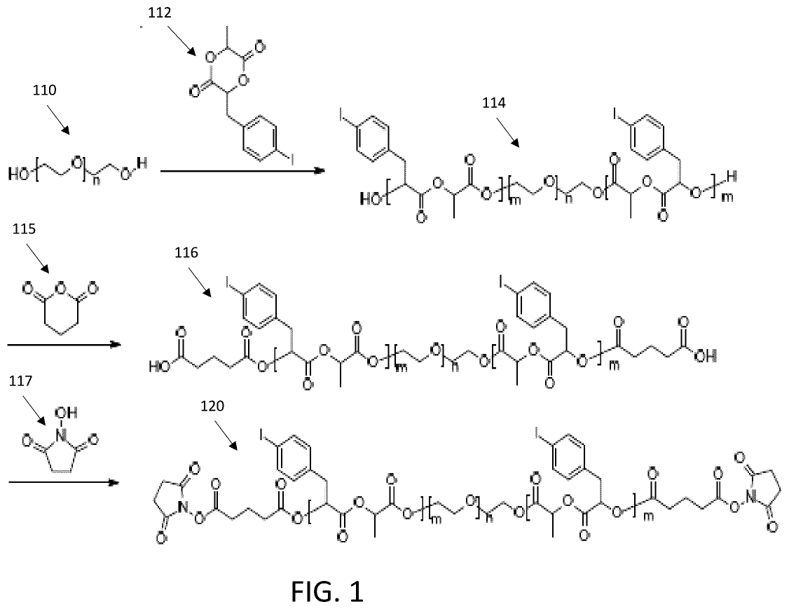

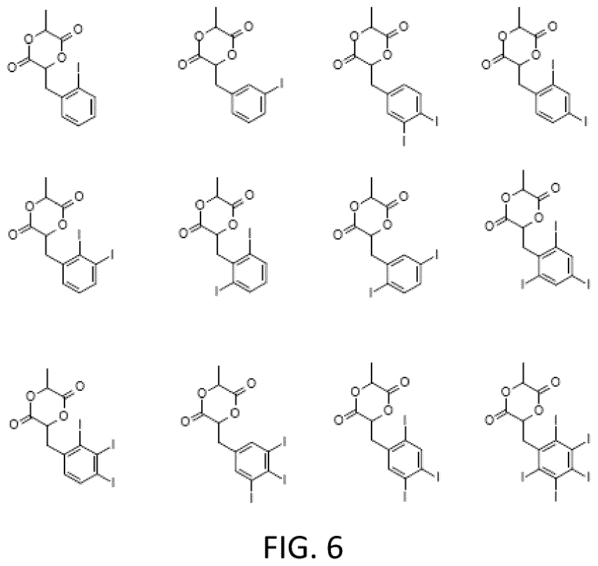

Iodinated cyclic monomers for use in the present disclosure include iodinated cyclic ester monomers. Iodinated cyclic ester monomers for use in the present disclosure include iodinated cyclic monoester monomers such as iodinated lactones (e.g., iodinated γ-butyrolactone, iodinated δ-valerolactone, iodinated ε-caprolactone, etc.). Iodinated cyclic ester monomers for use in the present disclosure further include iodinated cyclic diester monomers including iodinated 1,4-dioxane-2,5-dione compounds. 1,4-Dioxane-2,5-dione compounds include cyclic dimers of iodinated alpha hydroxy acids such as iodinated lactic acid or iodinated glycolic acid. In the particular example described in conjunction with FIG. 1, the iodinated monomer is an iodinated 1,4-dioxane-2,5-dione compound, specifically, 3-(4-iodobenzyl)-6-methyl-1,4-dioxane-2,5-dione. Further specific examples of iodinated 1,4-dioxane-2,5-dione monomers are shown in FIG. 6.

Iodinated cyclic monomers include cyclic monomers in which at least one ring carbon of a cyclic ester compound (e.g., selected from cyclic monoesters and cyclic diesters) is substituted with iodine or an iodinated moiety. Examples include cyclic ester compounds in which at least one ring carbon is substituted with an iodinated moiety that comprises an iodinated aromatic group. Examples of iodinated aromatic groups include iodine-substituted monocyclic aromatic groups and iodine-substituted multicyclic aromatic groups, such as iodinated phenyl groups, iodinated naphthyl groups, iodinated anthracenyl groups, iodinated phenanthrenyl groups, or iodinated tetracenyl groups. The iodinated aromatic groups may be substituted with one, two, three, four, five, six or more iodine atoms. In various embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups and hydroxyalkyl groups (e.g., hydroxyalkyl groups containing one carbon, two carbons, three carbons, four carbons, etc.). The iodinated aromatic groups may be directly linked to the ring carbon or may be linked to the ring carbon through any suitable linking moiety, which may be selected, for example, from an alkyl group, ether group, ester group, amide group, amine group, or carbonate group, among others

Specific examples of iodinated aromatic groups include those that comprise one or more monocyclic or multicyclic aromatic structures, substituted with (a) one or more iodine groups (e.g., one two, three, four, five, six or more iodine atoms) and (b) optionally, one or more hydroxyl-containing groups independently selected from one or more hydroxyl groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others, which C₁-C₄-hydroxyalkyl groups may be linked to the monocyclic or multicyclic aromatic structures directly or through any suitable linking moiety, which may be selected, for example, from alkyl groups, ether groups, ester groups, amide groups, amine groups, or carbonate groups, among others.

Ring-opening polymerization may be conducted by adding organic base (e.g., 1,8-diazabicyclo[5.4.0]undec-7-ene (DBU), triethylamine (TEA), etc.) or inorganic base (e.g., KOH, NaOH, K₂CO₃, etc.) with suitable feedstock of cyclic ester in a polar aprotic solvent such tetrahydrofuran (THF) at 50° C. and stirred overnight before quenching reaction by adding a saturated ammonium chloride aqueous solution.

Subsequent to ring-opening polymerization of the iodinated polymer segments, a reactive moiety is linked to the ends of the iodinated polymer segments. In various embodiments, the iodinated polymer segments either have hydroxyl end groups as a result of the ring-opening polymerization or can be modified to have hydroxyl end groups.

Such hydroxyl end groups are available for subsequent reactions, in particular, reactions that provide reactive moieties at the ends of the hydrophilic segments. Examples of such reactive moieties include reactive electrophilic moieties as described above. In particular embodiments the reactive moieties are selected from N-hydroxysuccinimide esters moieties, such as succinimidyl malonate moieties, succinimidyl glutarate moieties, succinimidyl succinate moieties, succinimidyl adipate moieties, succinimidyl diglycolate moieties or succinimidyl carbonate moieties, imidazole ester moieties, imidazole carboxylate moieties and/or benzotriazole ester moieties, among others.

In particular embodiments, terminal hydroxyl groups of the hydrophilic segments are reacted with a cyclic anhydride (e.g., glutaric anhydride, succinic anhydride, malonic anhydride, adipic anhydride, diglycolic anhydride, etc.) to form a reaction product that comprises a carboxylic acid group that is linked to the hydrophilic segment through a hydrolysable ester group, which is then treated with a coupling agent (e.g., a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) and N-hydroxysuccinimide (NHS), to yield a reactive succinimidyl end groups, in particular, a hydrolysable ester group and a reactive succinimidyl ester end group, such as a succinimidyl malonate group, succinimidyl glutarate group, succinimidyl succinate group, succinimidyl adipate group, or succinimidyl diglycolate group.

In a particular embodiment shown in FIG. 1 a commercially available hydroxy-terminated precursor linear hydrophilic polymer, specifically, bis-hydroxy-terminated PEG (110) is used as a bis-functional macro-initiator to react with 3-(4-iodobenzyl)-6-methyl-1,4-dioxane-2,5-dione (112) via ring-opening polymerization to produce a bis-hydroxy-terminated triblock copolymer (114) having a central PEG block and two iodinated polymer end blocks, more particularly two iodinated polyester end blocks, even more particularly two iodinated poly(lactic acid) (IPLA) end blocks in which the methyl side groups of half of the lactic acid residues are replaced with iodinated phenyl side groups (i.e., 4-iodo-benzyl side groups). Glutaric anhydride (CAS #108-55-4) (115) is then reacted with the bis-hydroxy-terminated IPLA-PEG-IPLA triblock copolymer (114) to obtain glutaric acid end-capped IPLA-PEG-IPLA triblock copolymer (116). Lastly, the glutaric acid end-capped IPLA-PEG-IPLA triblock copolymer (116) is reacted with n-hydroxysuccinimide (CAS #6066-82-6) (117) in the presence of a coupling agent to form an activated ester end-capped copolymer (120), specifically, a succinimidyl glutarate end-capped IPLA-PEG-IPLA triblock copolymer. In FIG. 1, n and m are integers. In certain embodiments, n may have a value ranging from 5 to 5000 and m may have a value ranging from 1 to 50.

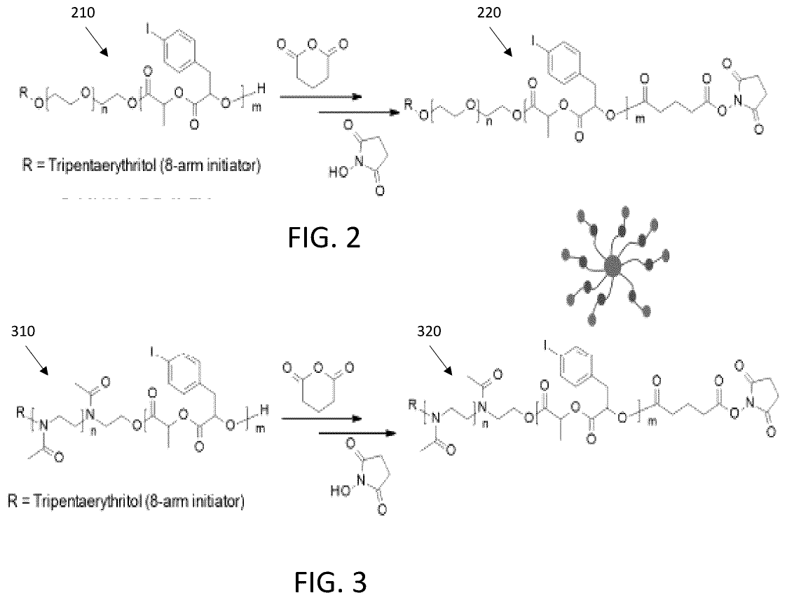

The strategy shown in FIG. 1 is widely applicable to other hydroxy-terminated hydrophilic polymers. For example, referring to FIG. 2 commercially available tripentaerythritol (CAS #78-24-0) can be used as an 8-arm initiator to undergo ring-opening polymerization with ethylene oxide to form hydroxy-terminated 8-arm-PEG. The hydroxy-terminated 8-arm-PEG is then used as an octa-functional macro-initiator to react with 3-(4-iodobenzyl)-6-methyl-1,4-dioxane-2,5-dione via ring-opening polymerization to produce a hydroxy terminated 8-arm copolymer (210) having a tripentaerythritol residue core and eight copolymer arms, each copolymer arm comprising a hydrophilic PEG segment linked to the core and a hydroxy-terminated iodinated poly(lactic acid) (IPLA) segment linked to each hydrophilic PEG segment. The 8-arm hydroxy terminated PEG-IPLA copolymer (210) is then reacted with glutaric anhydride to form to obtain a glutaric acid end-capped 8-arm PEG-IPLA copolymer (not shown), which is then reacted with n-hydroxysuccinimide in the presence of a coupling agent to form a succinimidyl glutarate end-capped 8-arm PEG-IPLA copolymer (220). In FIG. 2, n and m are integers. In certain embodiments, n may have a value ranging from 5 to 500 and m may have a value ranging from 1 to 100.

As another example, referring to FIG. 3 tripentaerythritol can be used as an 8-arm initiator to undergo ring-opening polymerization with 2-methyl-2-oxazoline to form hydroxy-terminated 8-arm-poly(2-methyl-2-oxazoline) (referred to herein as 8-arm POZ This hydroxy-terminated 8-arm-POZ is then used as an octa-functional macro-initiator to react with 3-(4-iodobenzyl)-6-methyl-1,4-dioxane-2,5-dione via ring-opening polymerization to produce a hydroxy terminated 8-arm copolymer (310) having a tripentaerythritol residue core and eight copolymer arms, each copolymer arm comprising a hydrophilic POZ segment linked to the core and a hydroxy-terminated iodinated poly(lactic acid) (IPLA) segment linked to each hydrophilic POZ segment. The 8-arm hydroxy terminated POZ-IPLA copolymer (310) is then reacted with glutaric anhydride to form to obtain a glutaric acid end-capped 8-arm POZ-IPLA copolymer (not shown), which is reacted with n-hydroxysuccinimide in the presence of a coupling agent to form a succinimidyl glutarate end-capped 8-arm POZ-IPLA copolymer (320). In FIG. 3, n and m are integers. In certain embodiments, n may have a value ranging from 5 to 500 and m may have a value ranging from 1 to 100.

It should be noted that although iodine groups are described, other radiopaque halogen groups including bromine may be employed.

In some aspects, the present disclosure provides a radiopaque hydrogel that comprises a crosslinked reaction product of (a) a radiopaque, reactive copolymer as described hereinabove (e.g., a radiopaque, reactive linear copolymer and/or a radiopaque, reactive multi-arm copolymer) and (b) a compound having a plurality of reactive nucleophilic moieties (e.g. amine moieties and/or thiol moieties, among others).

In particular embodiments, the compound having a plurality of reactive nucleophilic moieties is a polyamino compound. In general, polyamino compounds suitable for use in the present disclosure include, for example, small molecule polyamines (e.g., containing at least two amine groups, for instance, from 3 to 20 amine groups or more in certain embodiments), polymers having amine side groups, and branched polymers having amine end groups, including dendritic polymers having amine end groups. Polyamino compounds suitable for use in the present disclosure include those that comprises a plurality of —(CH₂)ₓ—NH₂ groups where x is 0, 1, 2, 3, 4, 5 or 6. Polyamino compounds suitable for use in the present disclosure include polyamino compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Particular examples of polyamino compounds which may be used as the polyamino compound include ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, and poly(allyl amine), among others among others.

In some embodiments, the polyamino compounds may be substituted with one or more radiopaque atoms such as iodine or bromine.

In various embodiments, the crosslinked reaction products of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values). Such crosslinked products may be formed in vivo (e.g., using a delivery device like that described below), or such crosslinked products may be formed ex vivo and subsequently administered to a subject. Such crosslinked products can be used in a wide variety of biomedical applications, including implants, medical devices, and pharmaceutical compositions.

In some aspects of the present disclosure, systems are provided that are configured to deliver (a) a polyamino compound (although a polyamino compound is described herein, it will be appreciated that other compounds having a plurality of reactive nucleophilic moieties may be employed) and (b) a radiopaque, reactive copolymer as described herein (e.g., a reactive linear copolymer and/or a radiopaque, reactive multi-arm copolymer). The polyamino compound and the radiopaque, reactive copolymer are combined under conditions such that the amino groups of the polyamino compound and the reactive moieties of the radiopaque, reactive copolymer crosslink with one another. In certain embodiments, those conditions comprise an environment having a basic pH, for example, a pH ranging from about 9 to about 11. Such systems can be used to form radiopaque crosslinked hydrogels, either in vivo or ex vivo.

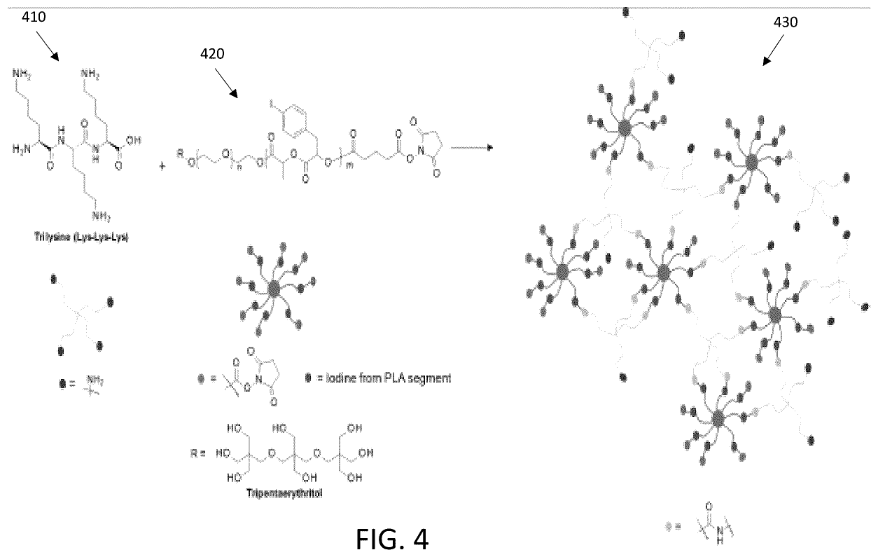

A particular example of a crosslinking reaction is shown in FIG. 4 which shows a succinimidyl glutarate end-capped 8-arm PEG-IPLA copolymer (420) like that of FIG. 2 being covalent crosslinked with trilysine (410) under basic conditions to form a radiopaque hydrogel product (430). In FIG. 4, each IPLA segment is short (e.g., ranging from 1 to 5 monomers in length) and m may have a value ranging from 1 to 5.

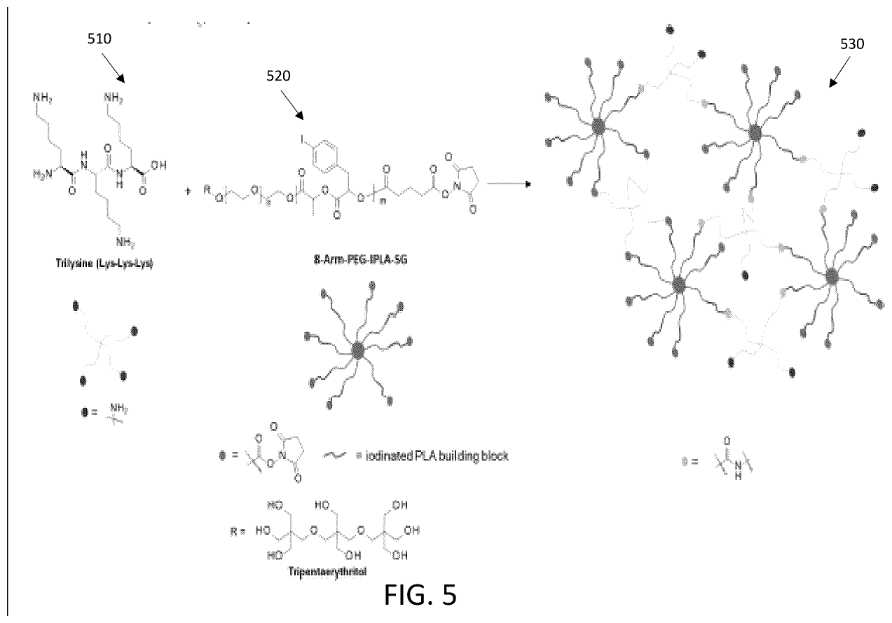

Another particular example of a crosslinking reaction is shown in FIG. 5 which shows a succinimidyl glutarate end-capped 8-arm PEG-IPLA copolymer (520) like that of FIG. 2 being covalent crosslinked with trilysine (510) under basic conditions to form a radiopaque hydrogel product (530). In FIG. 5, IPLA segment is longer (e.g., ranging from 6 to 50) and m has a value ranging from 6 to 50. Because the IPLA segment is longer, the resulting radiopaque hydrogel product (530) will have a greater radiopacity than the radiopaque hydrogel product (430) of FIG. 4.

In some aspects of the present disclosure, a system is provided that comprises (a) a first composition that comprises a polyamino compound, for example, as described herein (although a polyamino compound is described herein, it will be appreciated that other compounds having a plurality of reactive nucleophilic moieties may be employed) and (b) a second composition that comprises a radiopaque, reactive copolymer (e.g., a reactive linear copolymer and/or a radiopaque, reactive multi-arm copolymer) as described herein.

The first composition may be a first fluid composition comprising the polyamino compound or a first dry composition that comprises the polyamino compound, to which a suitable fluid such as water for injection, saline, etc. can be added to form a first fluid composition. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the radiopaque, reactive copolymer or a second dry composition that comprises the radiopaque, reactive copolymer, to which a suitable fluid such as water for injection, saline, etc. can be added to form a second fluid composition. In addition to the radiopaque, reactive copolymer, the second composition may further comprise additional agents, including therapeutic agents, imaging agents, colorants, tonicity adjusting agents, suspension agents, wetting agents, and pH adjusting agents as described below.

In some embodiments, the polyamino compound is initially combined with the radiopaque, reactive copolymer at an acidic pH at which crosslinking between the reactive moieties of the radiopaque, reactive copolymer and the amino groups of the polyamino compound is suppressed. Then, when crosslinking is desired, a pH of the mixture of the polyamino compound and the radiopaque, reactive copolymer is changed from an acidic pH to a basic pH, leading to crosslinking between same, thereby forming the crosslinked product.

In particular embodiments, the system comprises (a) a first composition that comprises a polyamino compound as described hereinabove, (b) a second composition that comprises a radiopaque, reactive copolymer as described hereinabove, and (c) a third composition, specifically, an accelerant composition, that contains an accelerant that is configured to accelerate a crosslinking reaction between the polyamino compound and the radiopaque, reactive copolymer.

The first composition may be a first fluid composition comprising the polyamino compound that is buffered to an acidic pH or a first dry composition that comprises the polyamino compound and acidic buffering composition, to which a suitable fluid such as water for injection, saline, etc. can be added to form a first fluid composition comprising the polyamino compound that is buffered to an acidic pH. In some embodiments, for example, the acidic buffering composition may comprise monobasic sodium phosphate, among other possibilities. The first fluid composition comprising the polyamino compound may have a pH ranging, for example, from about 3 to about 5. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the radiopaque, reactive copolymer or a second dry composition that comprises the radiopaque, reactive copolymer from which a fluid composition is formed, for example, by the addition of a suitable fluid such as water for injection, saline, or the first fluid composition comprising the polyamino compound that is buffered to an acidic pH. In addition to the radiopaque, reactive copolymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the polyamino compound that is buffered to an acidic pH and the second composition comprises a dry composition that comprises the radiopaque, reactive copolymer. The first composition may then be mixed with the second composition to provide a prepared fluid composition that is buffered to an acidic pH and comprises the polyamino compound and the radiopaque, reactive copolymer. In a particular example, a syringe may be provided that contains a first fluid composition comprising the polyamino compound that is buffered to an acidic pH, and a vial may be provided that comprises a dry composition (e.g., a powder) that comprises the radiopaque, reactive copolymer. The syringe may then be used to inject the first fluid composition into the vial containing the radiopaque, reactive copolymer to form a prepared fluid composition that contains the polyamino compound and the radiopaque, reactive copolymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents (beyond the radiopaque iodine atoms that are present) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

A prepared fluid composition that is buffered to an acidic pH and comprises the polyamino compound and the radiopaque, reactive copolymer as described above, and a fluid accelerant composition that is buffered to basic pH as described above, may be combined form radiopaque crosslinked hydrogels, either in vivo or ex vivo.

In various embodiments, a system is provided that include one or more delivery devices for delivering first and second compositions to a subject.

In some embodiments, the system may include a delivery device that comprises a first reservoir that contains a first composition that comprises a polyamino compound as described above and a second reservoir that contains a second composition that comprises a radiopaque, reactive copolymer that comprises a plurality of reactive moieties that are reactive with the amino moieties of the polyamino compound as described above.

In some embodiments, the system may include a delivery device that comprises a first reservoir that contains a first composition that comprises the polyamino compound and the radiopaque, reactive copolymer and is buffered to an acidic pH, such as the prepared fluid composition previously described, and a second reservoir that contains second composition, such as the fluid accelerant composition previously described. In either case, during operation, the first composition and second composition are dispensed from the first and second reservoirs and combined, whereupon the polyamino compound and the radiopaque, reactive copolymer and crosslink with one another to form a radiopaque crosslinked hydrogel.

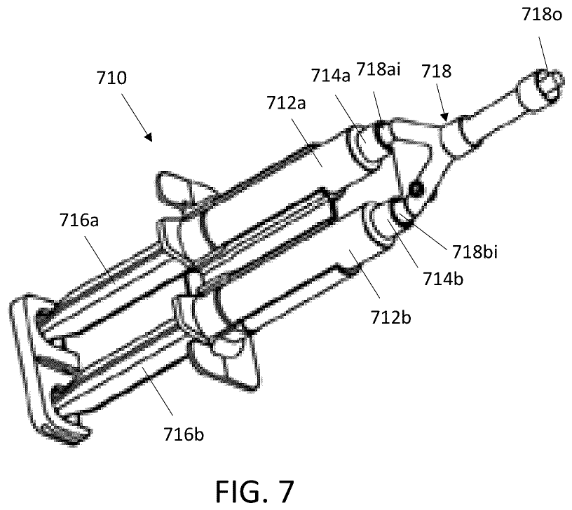

In particular embodiments, and with reference to FIG. 7, the system may include a delivery device 710 that comprises a double-barrel syringe, which includes first barrel 712a having a first barrel outlet 714a, which first barrel contains the first composition, a first plunger 716a that is movable in the first barrel 712a, a second barrel 712b having a second barrel outlet 714b, which second barrel 712b contains the second composition, and a second plunger 716b that is movable in the second barrel 712b. In some embodiments, the device 710 may further comprise a mixing section 718 having a first mixing section inlet 718ai in fluid communication with the first barrel outlet 714a, a second mixing section inlet 718bi in fluid communication with the second barrel outlet, and a mixing section outlet 7180.

In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a radiopaque crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a radiopaque crosslinked hydrogel is ultimately formed at the administration location.

After administration, the compositions of the present disclosure can be imaged using a suitable imaging technique. Typically, the imaging techniques is an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, radiopaque crosslinked hydrogels may be in any desired form, including a slab, a cylinder, a coating, or a particle. In some embodiments, the radiopaque crosslinked hydrogel is dried and then granulated into particles of suitable size. Granulating may be by any suitable process, for instance by grinding (including cryogrinding), crushing, milling, pounding, or the like. Sieving or other known techniques can be used to classify and fractionate the particles. Radiopaque crosslinked hydrogel particles formed using the above and other techniques may varying widely in size, for example, having an average size ranging from 50 to 950 microns.

In addition to a radiopaque crosslinked hydrogel as described above, radiopaque crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the radiopaque crosslinked hydrogel to a subject. Such systems may include one or more of the following: a syringe barrel, which may or may not contain a radiopaque crosslinked hydrogel as described herein; a vial, which may or may not contain a radiopaque crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the radiopaque crosslinked hydrogel may be provided in dry form (e.g., powder form) or in a form that is ready for injection, such as an injectable hydrogel form (e.g., a suspension of radiopaque crosslinked hydrogel particles).

The radiopaque crosslinked hydrogel compositions described herein can be used for a number of purposes.

For example, radiopaque crosslinked hydrogel compositions can be injected to provide spacing between tissues, radiopaque crosslinked hydrogel compositions can be injected (e.g., in the form of blebs) to provide fiducial markers, radiopaque crosslinked hydrogel compositions can be injected for tissue augmentation or regeneration, radiopaque crosslinked hydrogel compositions can be injected as a filler or replacement for soft tissue, radiopaque crosslinked hydrogel compositions can be injected to provide mechanical support for compromised tissue, radiopaque crosslinked hydrogel compositions be injected as a scaffold, and/or radiopaque crosslinked hydrogel compositions can be injected as a carrier of therapeutic agents in the treatment of diseases and cancers and the repair and regeneration of tissue, among other uses.

After administration, the radiopaque crosslinked hydrogel compositions of the present disclosure can be imaged using a suitable imaging technique.

As seen from the above, the radiopaque crosslinked hydrogel compositions of the present disclosure may be used in a variety of medical procedures, including the following, among others: a procedure to implant a fiducial marker comprising a radiopaque crosslinked hydrogel, a procedure to implant a tissue regeneration scaffold comprising a radiopaque crosslinked hydrogel, a procedure to implant a tissue support comprising a radiopaque crosslinked hydrogel, a procedure to implant a tissue bulking agent comprising a radiopaque crosslinked hydrogel, a procedure to implant a therapeutic-agent-containing depot comprising a radiopaque crosslinked hydrogel, a tissue augmentation procedure comprising implanting a radiopaque crosslinked hydrogel, a procedure to introduce a radiopaque crosslinked hydrogel between a first tissue and a second tissue to space the first tissue from the second tissue.

The radiopaque crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Radiopaque crosslinked hydrogel compositions in accordance with the present disclosure also include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A radiopaque, reactive copolymer comprising at least one hydrophilic polymer segment having a plurality of hydrophilic polymer segment ends, a plurality of iodinated polymer segments covalently linked to the plurality of hydrophilic polymer segment ends, and a plurality of reactive moieties covalently linked to the iodinated polymer segments.

2. The radiopaque, reactive copolymer of claim 1, wherein the at least one hydrophilic polymer segment comprises a single hydrophilic polymer segment, and the plurality of hydrophilic polymer segment ends comprise first and second ends of the single hydrophilic polymer segment.

3. The radiopaque, reactive copolymer of claim 2, wherein the first end of the single hydrophilic polymer segment is covalently linked to a first end of a first iodinated polymer segment of the plurality of iodinated polymer segments, the second end of the single hydrophilic polymer segment is covalently linked to a first end of a second iodinated polymer segment of the plurality of iodinated polymer segments, a first reactive moiety of the plurality of reactive moieties is covalently linked to a second end of the first iodinated polymer segment, and a second reactive moiety of the plurality of reactive moieties is covalently linked to a second end of the second iodinated polymer segment.

4. The radiopaque, reactive copolymer of claim 1, wherein the at least one hydrophilic polymer segment comprises a plurality of hydrophilic polymer segments and wherein the radiopaque, reactive copolymer comprises a plurality of polymer arms linked to a core region, the polymer arms each comprising one of the plurality hydrophilic polymer segments, one of the plurality of iodinated polymer segments, and one of the plurality of reactive moieties.

5. The radiopaque, reactive copolymer of claim 2, wherein a first end of the one of the plurality of hydrophilic polymer segments is covalently linked to the core region, a second end of the one of the plurality of hydrophilic polymer segments is covalently linked to a first end of the one of the plurality of iodinated polymer segments, and the one of the plurality of reactive moieties is covalently linked to a second end of the one of the plurality of iodinated polymer segments.

6. The radiopaque, reactive copolymer of claim 1, wherein the reactive moiety comprises an electrophilic group.

7. The radiopaque, reactive copolymer of claim 1, wherein the reactive moiety comprises a succinimide ester group, an imidazole ester group, an imidazole carboxylate group, or a benzotriazole ester group.

8. The radiopaque, reactive copolymer of claim 1, wherein the plurality of iodinated polymer segments each comprise a residue of one or more iodinated cyclic ester monomers.

9. The radiopaque, reactive copolymer of claim 8, wherein the iodinated cyclic ester monomer is selected from an iodinated lactone and an iodinated 1,4-dioxane-2,5-dione compound.

10. The radiopaque, reactive copolymer of claim 1, wherein the plurality of iodinated polymer segments is iodinated polyester segments in which at least one iodinated moiety is covalently attached to a polyester backbone of the iodinated polyester segments.

11. The radiopaque, reactive copolymer of claim 10, wherein the iodinated moiety comprises a monocyclic or multicyclic aromatic structure that is substituted with one or more iodine groups.

12. The radiopaque, reactive copolymer of claim 1, wherein the plurality of iodinated polymer segments each comprise a residue of an iodinated cyclic ester monomer, wherein an iodinated moiety is covalently attached to at least one ring carbon of a cyclic ester portion of the iodinated cyclic ester monomer.

13. The radiopaque, reactive copolymer of claim 12, wherein the monocyclic or multicyclic aromatic structure is further substituted with one or more hydroxyl groups and/or one or more C₁-C₄-hydroxyalkyl groups.

14. A system for forming a hydrogel composition that comprises (a) a nucleophilic compound and (b) a radiopaque, reactive copolymer comprising at least one hydrophilic polymer segment having a plurality of hydrophilic polymer segment ends, a plurality of iodinated polymer segments covalently linked to the plurality of hydrophilic polymer segment ends, and a plurality of reactive moieties covalently linked to the iodinated polymer segments.

15. The system of claim 14, wherein the nucleophilic compound is a polyamino compound.

16. The system of claim 15, wherein the system comprises a first composition that comprises the nucleophilic compound and a second composition that comprises the radiopaque, reactive polymer.

17. The system of claim 16, further comprising an accelerant composition.

18. The system of claim 14, further comprising a delivery device.

19. A radiopaque crosslinked hydrogel composition comprising a crosslinked reaction product of (a) a nucleophilic compound and (b) a radiopaque, reactive copolymer in accordance with claim 6.

20. A method of treatment comprising administering to a subject a mixture that comprises (a) a nucleophilic compound and (b) radiopaque, reactive copolymer comprising at least one hydrophilic polymer segment having a plurality of hydrophilic polymer segment ends, a plurality of iodinated polymer segments covalently linked to the plurality of hydrophilic polymer segment ends, and a plurality of reactive moieties covalently linked to the iodinated polymer segments, under conditions such that the nucleophilic compound and the radiopaque, reactive copolymer crosslink after administration.
